(19) United States
(12) Patent Application Publication
(10) Pub. No.: US 2026/0179873 A1
(43) Pub. Date: Jun. 25, 2026

(54) CLEANING SYSTEM FOR CLEANING AN OPTICAL ELEMENT OF A CHARGED PARTICLE EVALUATION SYSTEM

(71) Applicant: Applied Materials Israel Ltd., Rehovot (IL)

(72) Inventors: Sven Ruhle, Tel Aviv (IL); lrit Ruach Nir, Rehovot (IL); Rony Reuveni, Gedera (IL); Tal Shamai, Rehovot (IL); Dan Tuvia Fuchs, Rehovot (IL); Alexander Goldenshtein, Rehovot (IL); Shachar Faigenblat, Rehovot (IL)

(73) Assignee: Applied Materials Israel Ltd., Rehovot (IL)

(21) Appl. No.: 19/000,249

(22) Filed: Dec. 23, 2024

(51) Int. Cl. H01J 37/02; A61L 2/08; A61L 2/20; G02B 27/00; H01J 37/28

(52) U.S. Cl. CPC H01J 37/026; A61L 2/08; A61L 2/202; G02B 27/0006; H01J 37/28; A61L 2202/11; A61L 2202/14

(57) ABSTRACT

A cleaning system for cleaning an optical element of a vacuumed space of the charged particle evaluation system of a charged particle evaluation system, the cleaning system includes (a) an ozone supply system configured to provide a cleaning-related flow of ozone to the vacuumed space during a cleaning session; and (b) a laser beam optics configured to (i) generate a laser beam outside the vacuumed space, and (ii) direct the laser beam towards the cleaning-related flow of ozone thereby breaking the cleaning-related flow of ozone to provide cleaning elements for cleaning the optical element.

### BACKGROUND OF THE INVENTION

A sample may be evaluated by a charged particle evaluation system by scanning one or more regions of the sample with an electron beam. The sample may be a semiconductor wafer that includes multiple dies. Other samples may be provided.

The scanning of the one or more regions may unwantedly charge the sample and reduce the quality of the evaluation of the sample. The charging of the sample is reversed by illuminating the sample with a laser beam, using one or more optical elements that aggregate contamination.

There is a growing need to clean the one or more optical elements used for illuminating the sample by the laser beam.

### BRIEF SUMMARY OF THE INVENTION

According to an embodiment, there is provided a cleaning system for cleaning an optical element of a charged particle evaluation system, the cleaning system includes (a) an ozone supply system configured to provide a cleaning-related flow of ozone to a vacuumed space of the charged particle evaluation system during a cleaning session; wherein the optical element is positioned within the vacuumed space; and (b) a laser beam optics.

The laser beam optics is configured to (i) generate a laser beam outside the vacuumed space, and (ii) direct the laser beam towards the cleaning-related flow of ozone thereby breaking the cleaning-related flow of ozone to provide cleaning elements (such as oxygen radicals) for cleaning the optical element.

According to an embodiment, the optical element is a mirror used for deflecting the laser beam towards a sample during an electron beam based evaluation of the sample.

According to an embodiment, there is provided a method for cleaning an optical element of a column of a charged particle evaluation system, the method includes (a) providing a cleaning-related flow of ozone to a vacuumed space of the charged particle evaluation system during a cleaning session; (b) breaking the cleaning-related flow of ozone, using a laser beam generated outside the vacuumed space, to provide cleaning elements (such as oxygen radicals); and (c) cleaning the optical element by the cleaning elements.

### BRIEF DESCRIPTION OF THE DRAWINGS

The subject matter regarded as the invention is particularly pointed out and distinctly claimed in the concluding portion of the specification. The invention, however, both as to organization and method of operation, together with specimen s, features, and advantages thereof, may best be understood by reference to the following detailed description when read with the accompanying drawings in which:

FIG. 1 illustrates an example of a charged particle evaluation system and a cleaning system;

FIG. 2 illustrates an example of a charged particle evaluation system and a cleaning system;

FIG. 3 illustrates an example of a charged particle evaluation system and a cleaning system;

FIG. 4 illustrates an example of a charged particle evaluation system and a cleaning system;

FIG. 5 illustrates an example of a charged particle evaluation system and a cleaning system;

FIG. 6 illustrates an example of an ozone supply system of the cleaning system; and

FIG. 7 illustrates an example of a method.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

According to an embodiment there is provided a cleaning system for cleaning an optical element located within a vacuumed space of the charged particle evaluation system.

Examples of an optical element include a window, a mirror, a lens, a filter and the like. For simplicity of explanation it is assumed that thew optical element is a mirror. Any reference to a mirror should be applied mutatis mutandis to any other optical element.

According to an embodiment, the cleaning system includes an ozone supply system configured to provide a cleaning-related flow of ozone to the vacuumed space of the charged particle evaluation system during a cleaning session. The optical element is positioned within the vacuumed space. The cleaning system further includes a laser beam optics configured to (i) generate a laser beam outside the vacuumed space, and (ii) direct the laser beam towards the cleaning-related flow of ozone thereby breaking the cleaning-related flow of ozone to provide cleaning elements (such as oxygen radicals) for cleaning the optical element.

According to an embodiment the optical element is a mirror used for deflecting the laser beam towards a sample during an electron beam based evaluation of the sample. According to an embodiment, the laser beam is deflected and then impinges on the sample and discharges the sample.

According to an embodiment, the vacuumed space is located within a column of the charged particle evaluation system.

According to an embodiment, the vacuumed space is located within a vacuum chamber of the charged particle evaluation system- and outside the column.

According to an embodiment, using the laser beam optics for discharging the sample and for cleaning reduces the cost and the complexity of the cleaning system.

It has been found that the energy of the laser photons required to break the oxygen-oxygen bond of the ozone to provide cleaning elements (such as oxygen radicals) for cleaning the optical elements is about ten percent lower than the photon energy required to break the oxygen-oxygen bond in an oxygen molecule. Assuming cleaning elements that are (such as oxygen radicals)—the oxygen radicals react and decompose organic contamination on optical elements.

In a vacuum system it has been found that a low partial pressure of ozone down to 10-5 Torr or less is sufficient-which allows to maintain a high vacuum level within the chamber of the charged particle evaluation system. Using ozone as an oxygen radical source improves the cleaning speed by an order of magnitude compared to oxygen and clean dry air (CDA).

According to an embodiment, cleaning sessions are triggered in a periodic manner or in a non-periodic manner, in a random manner, in a pseudo random manner, based on one or more parameters of the charged particle evaluation system such as availability, or in any other predefined manner.

According to an embodiment, cleaning sessions are triggered based on an analysis of a quality of the evaluation—for example by analyzing images acquired by the charged particle evaluation system.

According to an embodiment, cleaning sessions are triggered based on the power of the laser beam—as higher contamination levels are associated with lower power levels of the laser beam.

According to an embodiment, the cleaning system includes a monitoring system that is configured to measure the power of the laser beam to monitor contamination of the optical element. The monitoring system includes one or more processing circuits and one or more hardware memory resources.

According to an embodiment, the cleaning system includes a controller that is configured to trigger the cleaning session when detecting at least a defined amount of contamination. The controller includes one or more processing circuits and one or more hardware memory resources.

According to an embodiment, the cleaning system includes a monitoring system mirror that is selectively optically coupled to the optical element and is configured to direct the laser beam towards a sensor of the monitoring system.

According to an embodiment, the cleaning system or the charged particle evaluation system includes a movement mechanism that is configured to optically couple the optical element to the monitoring system by introducing a relative movement between a column of the charged particle evaluation system and a mechanical stage that supports the sample during the electron beam based evaluation.

According to an embodiment, cleaning system includes an ozone source that is configured to generate an initial flow of ozone.

According to an embodiment, the cleaning-related flow of ozone is the initial flow of ozone.

According to an embodiment, the cleaning-related flow of ozone is a first portion of the initial flow of ozone.

According to an embodiment, the cleaning system also includes an extermination branch that is configured to receive a second portion of the initial flow of ozone.

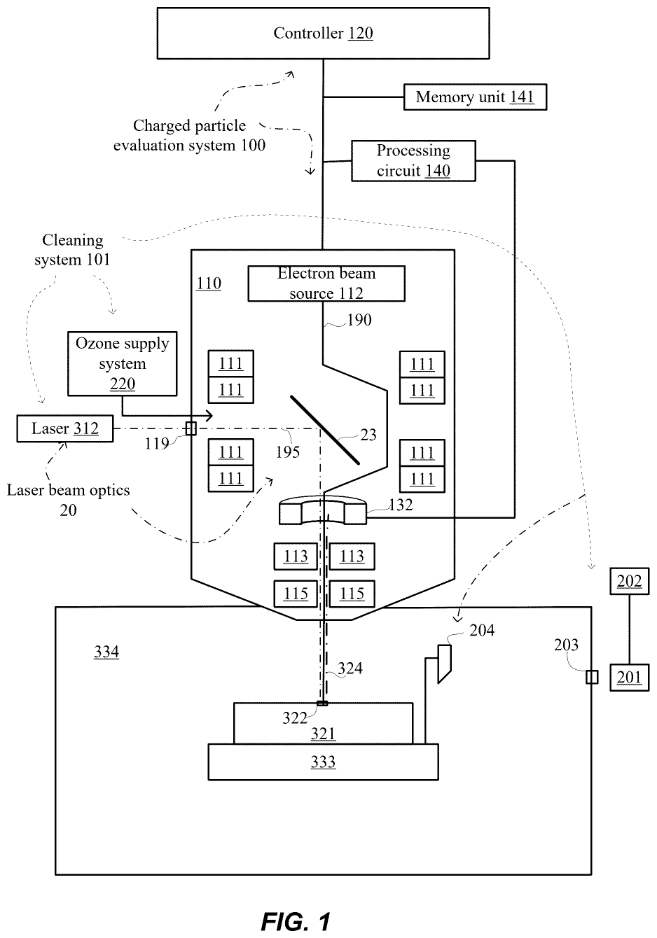

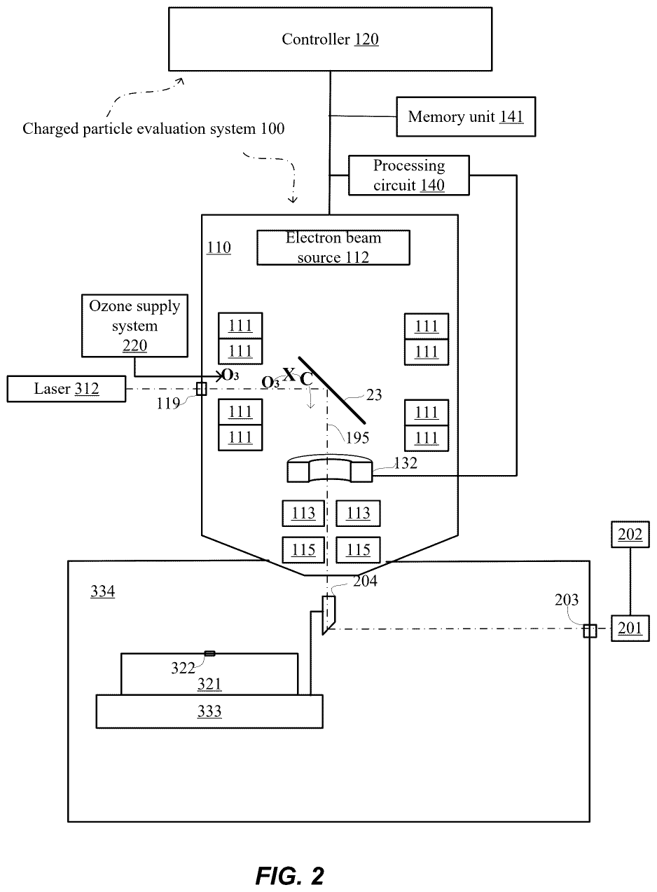

FIGS. 1 and 2 illustrate an example of a charged particle evaluation system 100, cleaning system 101, and sample 321. In FIGS. 1 and 2 the optical element is located within the column 110 of the charged particle evaluation system.

According to an embodiment, charged particle evaluation system 100 is one of (i) a defect review charged particle evaluation system SEMVISION™ of APPLIED MATERIALS™ Inc. of San Jose, California, (ii) a metrology system such as the PROVision™ 3E Ebeam™ metrology system of APPLIED MATERIALS™, (iii) an electron beam inspection system such as the PRIMEVISION™ of APPLIED MATERIALS™, or (iv) a critical dimension charged particle evaluation system such as the VERITYSEM™ of APPLIED MATERIALS™, and the like. The charge particle evaluation system may be manufactured by vendors such as HITACHI™ of Tokyo, Japan, or KLA™ Corporation of Milpitas, California, or may be manufactured by other vendors.

FIG. 1 illustrates the charged particle evaluation system 100 and the cleaning system 101 during an evaluation of the sample, having an electron beam 190 that illuminates region 322 of sample 321, having electrons 324 from the region reach secondary electron sensor 132, and having laser beam 195 directed towards region 322 in order to reduce charging of the sample by the electron beam 190.

FIG. 2 illustrates the charged particle evaluation system 100 and the cleaning system 101 during a cleaning session iteration in which the electron beam is not generated (or is generated and not directed towards the region), laser beam 195 and ozone supply system 220 are used to clean an optical element such as mirror 23, and monitoring system mirror 204 is selectively optically coupled to the mirror 23 and is configured to direct the laser beam through additional window 203 form in vacuum chamber 334 towards a sensor 201 of the monitoring system. The sensor 201 of the monitoring system generates laser beam power signals that are processed by cleaning system controller 202 in order to determine whether to trigger a cleaning session. The cleaning system controller 202 is also configured to trigger cleaning sessions regardless of the measurement made by the measurement system. Ozone (Denoted O₃) is broken by the laser beam to provide cleaning elements (denoted X) that interact with contamination (Denoted C) thereby removing the contamination from the mirror.

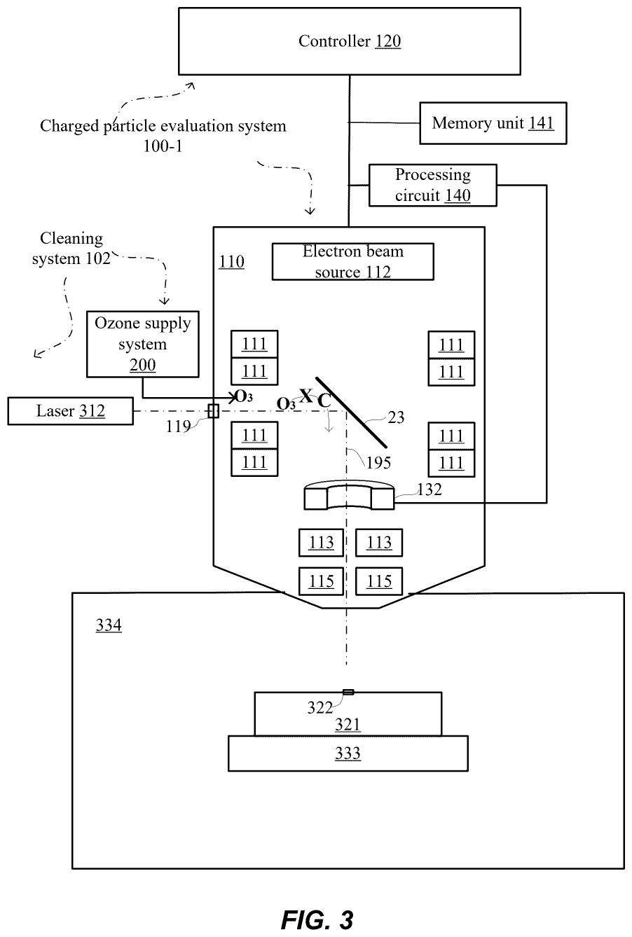

FIG. 3 illustrates a cleaning session executed by a cleaning system 102 that differs from cleaning system 101 of FIGS. 1 and 2 by not having the monitoring system.

The charged particle evaluation system 100 uses charged particles to evaluate a sample.

FIGS. 1-3 illustrate laser beam optics 20 as including:
a. Laser 312 is located outside the column 110 of the charged particle evaluation system and is configured to generate a laser beam.
b. Mirror 23 is located within the column 110 and is configured to receive the laser beam through a window 119 that is formed in the column 110, and to direct the laser beam towards region 322 that is scanned by electron beam 190, during one or more illumination iterations.

The laser beam optics 20 may include one or more additional optical components such as focusing lenses, collimators, polarization control elements, and the like.

According to an embodiment the laser beam is continuous or pulsed.

The charged particle evaluation system 100 includes:
a. Column 110.
b. Memory unit 141 for storing instructions and data.
c. Controller 120 is configured to generate control signals that control the scanning of region 322 by electron beam 190. Different regions are scanned by introducing, under the control of controller 120, a relative movement between the sample and the column.
d. A sensing unit that includes one or more sensors such as secondary electron sensor 132 located within column 110.
e. A processing circuit 140 that is configured to convert sensor signals to frames and to images. The processing circuit 140 may include an array of integrated circuits such as graphic processors, general purpose processors, and the like.
f. Vacuum chamber 334 in which the sample is located.
g. Mechanical stage 333 for supporting the sample and for moving the sample.

According to an embodiment, the one or more detectors of the sensing unit may include one or more secondary electron detectors, one or more backscattered electron detectors, and the like. Detectors for detecting photons and additionally or alternatively x-rays may also be included in the charged particle evaluation system. Examples of a column 110 that includes multiple sensors are illustrated in U.S. Pat. No. 7,847,267 of Shemesh et al., which is incorporated herein by reference for all purposes. Any detector of the charged particle evaluation system may be located within the column or outside the column. A detector may include a single sensing segment, may include multiple sensing segments, the detector may be a part of an array of sensors, and the like.

According to an embodiment, column 110 includes electron optics such as electron beam source 112 and electron beam manipulation optics that is configured to propagate the electron beam 190 through the column (for example, while bypassing mirror 23) till exiting the column.

The electron beam manipulation optics may include deflection lenses, focusing lenses, electron beam collimating optics, electron beam shaping optics, and the like. Examples of a column 110 that includes multiple deflection coils for double deflecting an electron beam are illustrated in U.S. Pat. No. 7,847,267 of Shemesh et al.

FIGS. 1-3 illustrate the electron beam manipulation optics as including:
a. Bypass magnetic scan coils 111 that are configured to direct the electron beam along a bypass path that bypasses mirror 23.
b. Objective lens 113 is configured to focus the electron beam on the region 322 of sample 321.
c. Deflector lenses 115 for deflecting the electron beam according to a scan pattern.

According to an embodiment, any other electron beam manipulation optics is provided—for example the electron beam may not be double-deflected within the column.

Referring back to FIGS. 1-3, an optical axis of the electron beam 190 is a vertical axis through which the electron beam propagated before the bypassing of the mirror and after the bypassing of the mirror.

The bypass magnetic scan coils 111 are configured to: (i) tilt the electron beam at a first direction, (ii) tilt the electron beam at an opposite direction such as to propagate along a secondary optical axis that is parallel to the optical axis but spaced apart from the optical axis, (iii) tilt the electron beam at a second direction, towards the optical axis, and (iv) tilt the electron beam, at a direction opposing the second direction, such as to propagate along the optical axis. A system and method for double tilt is described at U.S. Pat. No. 6,674,075 of Petrov et al., and is incorporated herein by reference for all purposes.

The charged particle evaluation system may also include a vacuum system (not shown) configured to maintain the column is maintained in vacuum, a high power supply unit (not shown) configured to provide high voltage signals to accelerate the electron beam and to decelerate the electron beam.

According to an embodiment, the mechanical stage 333 is used for moving the monitoring system mirror 204 (that is mechanically coupled to the mechanical stage 33) to be aligned with the laser beam reflected from the mirror—as illustrated in FIG. 2.

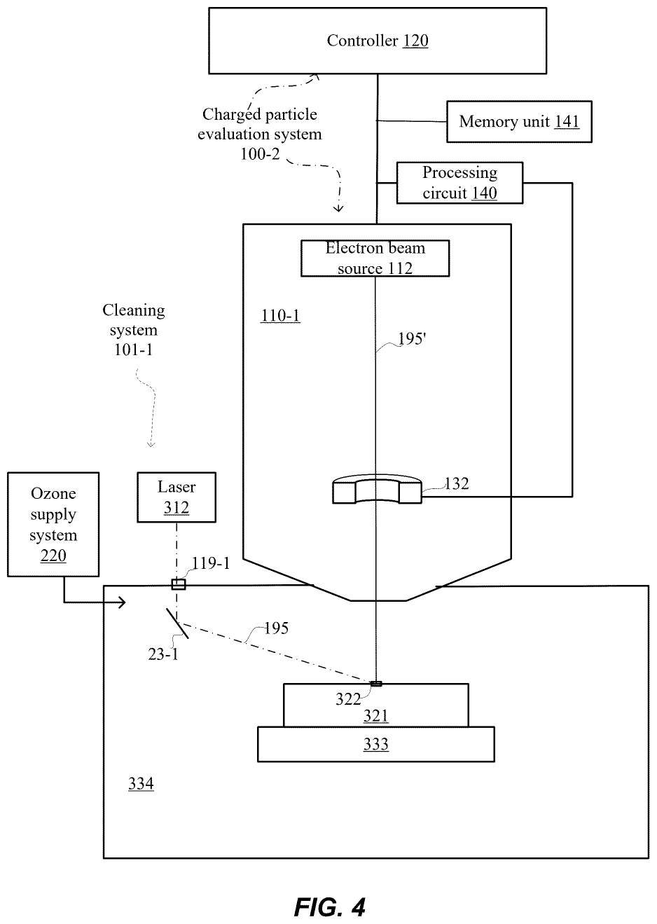

FIG. 4 illustrates an example of charged particle evaluation system 100-1, cleaning systems 101-1, and sample 321. In FIG. 4, mirror 23-1 is located within the vacuum chamber 334 and outside the column 110-1 which is illustrated, for simplicity of examination without various elements illustrated in FIGS. 1 and 2. Mirror 23-1 is configured to deflect a laser beam 195 that enters the vacuum chamber 334 through additional window 119-1. The laser beam is deflected towards the sample- and especially towards region 322 of sample 321.

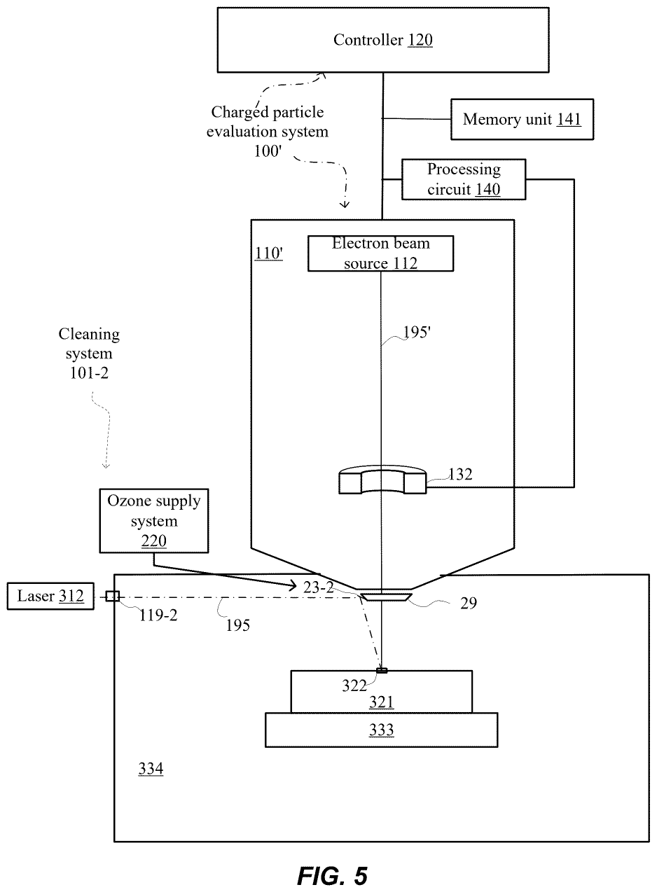

For simplicity of explanation, a monitoring system was not illustrated in FIGS. 4 and 5. Any cleaning system of FIG. 4 or 5 may or may not include a monitoring system.

FIG. 5 illustrates an example of charged particle evaluation system 100′, cleaning systems 101-2, and sample 321. In FIG. 5, mirror 23-2 is located within the vacuum chamber 334 and outside the column. The mirror 23-2 is a part of an electrode 29 of the charged particle evaluation system 100-2 or is mechanically and electrically coupled to electrode 92. Mirror 23-2 is configured to deflect a laser beam 195 that enters the vacuum chamber 334 through further window 119-2. The laser beam is deflected towards the sample- and especially towards region 322 of sample 321. Examples of mirrors that belong to such as electrode or are coupled to the electrode are illustrated in U.S. Pat. No. 9,673,023, which is incorporated herein by reference.

FIG. 5 illustrates column 110′ as not deflecting the electron beam 195′. According to an embodiment, the column of any of the charged particle evaluation systems illustrated in any of the figures is configured to manipulate the electron beam in any manner—for example perform a double deflection or preform a single deflection or perform three or more deflections.

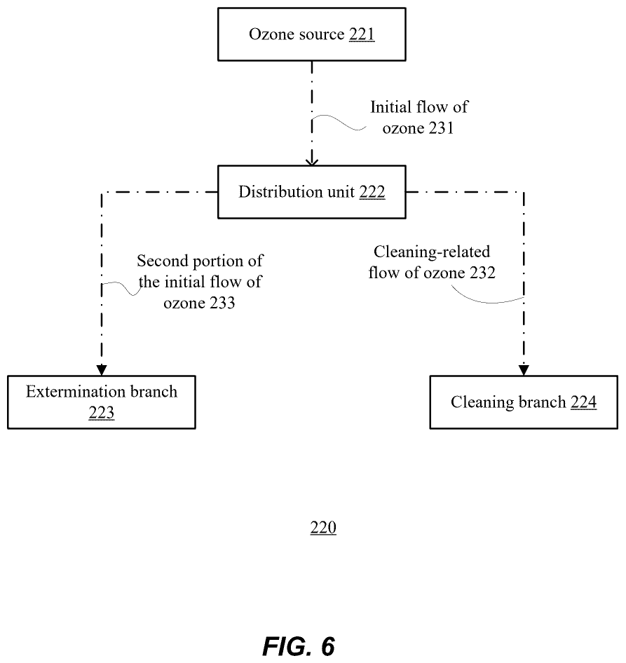

FIG. 6 illustrates an example of an ozone supply system 200 of the cleaning system.

According to an embodiment, the ozone supply system 200 includes:
a. Ozone source 221 is configured to generate an initial flow of ozone 231.
b. Distribution unit 222 configured to split the initial flow of ozone to a first portion, which is the cleaning-related flow of ozone 232, and to a second portion of the initial flow of ozone 233.
c. A cleaning branch 224 that provides the cleaning-related flow of ozone 232 to the charged particle evaluation system, for example, to the vacuum chamber 334, to column 110 or 110′, and the like.

d. An extermination branch 223 that is configured to receive the second portion of the initial flow of ozone 233 and exterminate the ozone, for example, by converting the ozone to another element.

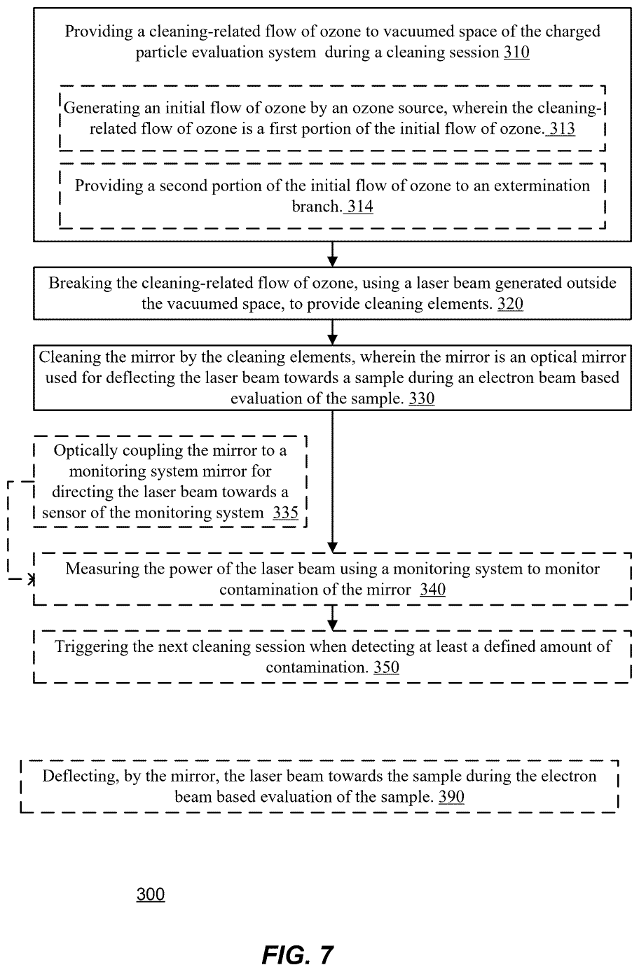

FIG. 7 illustrates an example of method 300 for cleaning an optical element of a charged particle evaluation system.

According to an embodiment, method 300 includes step 310 of providing a cleaning-related flow of ozone to a vacuumed space of the charged particle evaluation system during a cleaning session. The optical element is located within the vacuumed space.

According to an embodiment, step 310 is followed by step 320 of breaking the cleaning-related flow of ozone, using a laser beam generated outside the vacuumed space, to provide cleaning elements.

According to an embodiment, step 320 is followed by step 330 of cleaning the optical element by the cleaning elements.

According to an embodiment, the optical element is a mirror used for deflecting the laser beam towards a sample during an electron beam based evaluation of the sample.

According to an embodiment, method 300 includes step 340 of measuring the power of the laser beam using a monitoring system to monitor contamination of the optical element.

According to an embodiment, step 340 is followed by step 350 of triggering the next cleaning session when detecting at least a defined amount of contamination.

According to an embodiment, step 340 is preceded by step 335 of optically coupling the optical element to a monitoring system mirror for directing the laser beam towards a sensor of the monitoring system.

According to an embodiment, step 335 includes introducing a relative movement between a column of the charged particle evaluation system and the mechanical stage that supports the sample during the electron beam based evaluation.

According to an embodiment, step 310 includes step 313 of generating an initial flow of ozone by an ozone source.

According to an embodiment, the cleaning-related flow of ozone is a first portion of the initial flow of ozone.

According to an embodiment, and when the cleaning-related flow of ozone is the first portion of the initial flow of ozone, step 313 is followed by step 314 of providing a second portion of the initial flow of ozone to an extermination branch.

According to an embodiment, the optical element is a mirror and method 300 also include step 390 of deflecting, by the mirror, the laser beam towards the sample during the electron beam based evaluation of the sample.

In the foregoing detailed description, numerous specific details are set forth in order to provide a thorough understanding of the embodiments of the disclosure.

However, it will be understood by those skilled in the art that the present embodiments of the disclosure may be practiced without these specific details. In other instances, well-known methods, procedures, and components have not been described in detail so as not to obscure the present embodiments of the disclosure.

The subject matter regarded as the embodiments of the disclosure is particularly pointed out and distinctly claimed in the concluding portion of the specification. The embodiments of the disclosure, however, both as to organization and method of operation, together with objects, features, and advantages thereof, may best be understood by reference to the following detailed description when read with the accompanying drawings.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

Because the illustrated embodiments of the disclosure may for the most part, be implemented using optical and/or electronic components and circuits known to those skilled in the art, details will not be explained in any greater extent than that considered necessary as illustrated above, for the understanding and appreciation of the underlying concepts of the present embodiments of the disclosure and in order not to obfuscate or distract from the teachings of the present embodiments of the disclosure.

Any reference in the specification to a method should be applied mutatis mutandis to a system capable of executing the method and should be applied mutatis mutandis to a computer program product that stores instructions that once executed result in the execution of the method.

Any reference in the specification to a system should be applied mutatis mutandis to a method that may be executed by the system should be applied mutatis mutandis to a computer program product that stores instructions that can be executed by the system.

Any reference in the specification to a computer program product should be applied mutatis mutandis to a method that may be executed when executing instructions stored in the computer program product and should be applied mutandis to a system that is configured to executing instructions stored in the computer program product.

The term and/or means additionally or alternatively. For example, A and/or B means only A, or only B or A and B.

In the foregoing specification, the embodiments of the disclosure have been described with reference to specific examples of embodiments. It will, however, be evident that various modifications and changes may be made therein without departing from the broader spirit and scope of the appended claims.

Moreover, the terms “front,” “back,” “top,” “bottom,” “over,” “under” and the like in the description and in the claims, if any, are used for descriptive purposes and not necessarily for describing permanent relative positions. It is understood that the terms so used are interchangeable under appropriate circumstances such that the embodiments of the disclosure described herein are, for example, capable of operation in other orientations than those illustrated or otherwise described herein.

Any reference to the term “comprising” or “having” or “including” should be applied mutatis mutandis to “consisting” and additionally or alternatively should be applied mutatis mutandis to “consisting essentially of”.

Any arrangement of components to achieve the same functionality is effectively “associated” such that the desired functionality is achieved. Hence, any two components herein combined to achieve a particular functionality may be seen as “associated with” each other such that the desired functionality is achieved, irrespective of architectures or intermedial components. Likewise, any two components so associated can also be viewed as being “operably connected,” or “operably coupled,” to each other to achieve the desired functionality.

Furthermore, those skilled in the art will recognize that boundaries between the above-described operations merely illustrative. The multiple operations may be combined into a single operation, a single operation may be distributed in additional operations and operations may be executed at least partially overlapping in time. Moreover, alternative embodiments may include multiple instances of a particular operation, and the order of operations may be altered in various other embodiments.

Also, for example, in one embodiment, the illustrated examples may be implemented as circuitry located on a single integrated circuit or within a same device. Alternatively, the examples may be implemented as any number of separate integrated circuits or separate devices interconnected with each other in a suitable manner.

Also, for example, the examples, or portions thereof, may implemented as soft or code representations of physical circuitry or of logical representations convertible into physical circuitry, such as in a hardware description language of any appropriate type.

However, other modifications, variations and alternatives are also possible. The specifications and drawings are, accordingly, to be regarded in an illustrative rather than in a restrictive sense.

In the claims, any reference signs placed between parentheses shall not be construed as limiting the claim. The word ‘comprising’ does not exclude the presence of other elements or steps then those listed in a claim. Furthermore, the terms “a” or “an,” as used herein, are defined as one or more than one. Also, the use of introductory phrases such as “at least one” and “one or more” in the claims should not be construed to imply that the introduction of another claim element by the indefinite articles “a” or “an” limits any particular claim containing such introduced claim element to embodiments containing only one such element, even when the same claim includes the introductory phrases “one or more” or “at least one” and indefinite articles such as “a” or “an.” The same holds true for the use of definite articles. Unless stated otherwise, terms such as “first” and “second” are used to arbitrarily distinguish between the elements such terms describe. Thus, these terms are not necessarily intended to indicate temporal or other prioritization of such elements. The mere fact that certain measures are recited in mutually different claims does not indicate that a combination of these measures cannot be used to advantage.

While certain features of the embodiments have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A cleaning system for cleaning an optical element of a charged particle evaluation system, the cleaning system comprising:
an ozone supply system configured to provide a cleaning-related flow of ozone to a vacuumed space of the charged particle evaluation system during a cleaning session, wherein the optical element is positioned within the vacuumed space; and
a laser beam optics configured to: (i) generate a laser beam outside the vacuumed space, and (ii) direct the laser beam towards the cleaning-related flow of ozone thereby breaking the cleaning-related flow of ozone to provide cleaning elements for cleaning the optical element.

2. The cleaning system according to claim 1, further comprising a monitoring system that is configured to measure a power of the laser beam to monitor contamination of the optical element.

3. The cleaning system according to claim 2, further comprising a controller that is configured to trigger the cleaning session when detecting at least a defined amount of the contamination of the optical element.

4. The cleaning system according to claim 3, further comprising a monitoring system mirror that is selectively optically coupled to the optical element and is configured to direct the laser beam towards a sensor of the monitoring system.

5. The cleaning system according to claim 2, further comprising a movement mechanism that is configured to optically couple the optical element to the monitoring system by introducing a relative movement between a column of the charged particle evaluation system and a mechanical stage that supports a sample during an electron beam based evaluation of the sample.

6. The cleaning system according to claim 1, further comprising an ozone source that is configured to generate an initial flow of ozone.

7. The cleaning system according to claim 6, wherein the cleaning-related flow of ozone is a first portion of the initial flow of ozone.

8. The cleaning system according to claim 7, further comprising an extermination branch that is configured to receive a second portion of the initial flow of ozone.

9. The cleaning system according to claim 1, wherein the optical element is a mirror used for deflecting the laser beam towards a sample during an electron beam based evaluation of the sample.

10. A method for cleaning an optical element of a column of a charged particle evaluation system, the method comprising:
providing a cleaning-related flow of ozone to a vacuumed space of the charged particle evaluation system during a cleaning session;
breaking, using a laser beam generated outside the vacuumed space, the cleaning-related flow of ozone to provide cleaning elements; and
cleaning the optical element by the cleaning elements.

11. The method according to claim 10, comprising measuring a power of the laser beam using a monitoring system to monitor contamination of the optical element.

12. The method according to claim 11, comprising triggering the cleaning session when detecting at least a defined amount of the contamination of the optical element.

13. The method according to claim 11, wherein the measuring of the power of the laser beam is preceded by optically coupling the optical element to a monitoring system mirror for directing the laser beam towards a sensor of the monitoring system.

14. The method according to claim 13, wherein the optically coupling of the optical element comprises introducing a relative movement between a column of the charged particle evaluation system and a mechanical stage that supports a sample during an electron beam based evaluation of the sample.

15. The method according to claim 10, further comprises generating an initial flow of ozone by an ozone source.

16. The method according to claim 15, wherein the cleaning-related flow of ozone is a first portion of the initial flow of ozone.

17. The method according to claim 16, further comprising providing a second portion of the initial flow of ozone to an extermination branch.

18. The method according to claim 11, wherein the optical element is a mirror.

19. The method according to claim 18, further comprising deflecting, by the mirror, the laser beam towards a sample during an electron beam based evaluation of the sample.
